(19) United States
(12) Patent Application Publication
(10) Pub. No.: US 2023/0072035 A1
(43) Pub. Date: Mar. 9, 2023

(54) ELECTROCHEMICAL SENSOR SYSTEM AND METHOD FOR ASCORBIC ACID MEASUREMENT

(71) Applicant: Purdue Research Foundation, West Lafayette, IN (US)

(72) Inventor: Wenzhuo Wu, West Lafayette, IN (US)

(21) Appl. No.: 17/849,840

(22) Filed: Jun. 27, 2022

Related U.S. Application Data
(60) Provisional application No. 63/237,814, filed on Aug. 27, 2021.

(51) Int. Cl. A61B 5/145; G01N 27/327; A61B 5/1477; A61B 5/00

(52) U.S. Cl. CPC A61B 5/14546; G01N 27/3278; A61B 5/1477; A61B 5/6801

(57) ABSTRACT

An electrochemical sensor that includes a substrate and a piezoelectric semiconductor which is configured to detect ascorbic acid using piezo-electrocatalysis. The piezoelectric semiconductor is coupled to the substrate. The piezoelectric semiconductor includes a nanostructured semiconducting ZnO catalyst. The nanostructured semiconducting ZnO catalyst has a noncentrosymmetric wurtzite configuration. The nanostructured semiconducting ZnO catalyst is shaped as a nanorod and/or a nanosheet.

### CROSS REFERENCE TO RELATED APPLICATIONS

This application claims the benefit of U.S. Provisional Application Ser. No. 63/237,814, filed on Aug. 27^{th}, 2021. The entire disclosure of the above application is hereby incorporated herein by reference.

### GOVERNMENT RIGHTS

This invention was made with government support under DE-AC07-05ID14517 awarded by the Department of Energy. The government has certain rights in the invention.

### FIELD

The present disclosure relates to sensors and, more particularly, to electrochemical sensors.

### INTRODUCTION

This section provides background information related to the present disclosure which is not necessarily prior art.

L-ascorbic acid (AA) is a critical nutrient for many organisms and an acidity regulator of antioxidants and preservatives. AA plays a vital role in biological metabolisms, e.g., the digestion of amino acids, as well as the synthesis of adrenalin, certain hormones, and neurotransmitters. AA has also been commonly used for the prevention and treatment of scurvy, cancer, common cold, and AIDS, etc. The design and implementation of cost-effective, high-performance electrochemical sensors for the rapid and accurate quantification of AA concentration in foods or biological fluids are important for many societally pervasive applications such as clinical diagnostics, wearable health monitoring, food safety, and environmental monitoring. However, the direct electro-oxidation of AA on the surface of the bare electrodes is irreversible. Moreover, the subsequent hydrolysis of the reaction will cause electrode fouling with large overpotential, poor selectivity, low sensitivity, and unsatisfactory reproducibility.

Nanostructured catalysts with large specific surfaces and abundant active sites appeal to highly sensitive electrochemical sensing of various chemicals. Various nanomaterials, such as conducting polymers, carbon materials, and metal oxides, have been explored for modifying the electrodes to efficiently detect AA, leveraging the enhanced active sites, improved interfacial charge transfer, and/or intrinsic conductivity in the related nanostructures. For example, Jiang et al. used liquid-phase exfoliated graphene to fabricate an AA sensor, which showed a linear detection range from 9 μM to 2314 μM with a detection limit of 6.45 μM. Recently, Mei et al. demonstrated the sensitive detection of AA with metal oxide nanomembranes and achieved a detection range of 1-30 μM. However, the preparation of these nanoengineered catalysts is complicated, usually involving high temperature or strict gas control. The catalyst yield is usually low, and the catalyst is also prone to be oxidized, reduced, or decomposed. Meanwhile, due to the use of expensive materials such as precious metals, biomaterials, complex instruments, and harsh control conditions, the cost of producing nanostructured AA catalysts is typically high. The design and implementation of cost-effective, high-performance electrochemical sensors for detecting AA remain a significant challenge.

### SUMMARY

In concordance with the instant disclosure, a cost-effective, high-performance electrochemical sensor system and method for detecting AA, has been surprisingly discovered.

The sensor is configured to detect ascorbic acid using piezo-electrocatalysis. The sensor includes a substrate and a piezoelectric semiconductor. The piezoelectric semiconductor may be coupled to the substrate. The piezoelectric semiconductor may also include a nanostructured semiconducting zinc oxide catalyst. In certain circumstances, the nanostructured semiconducting zinc oxide catalyst may have a noncentrosymmetric wurtzite configuration.

In another embodiment, the present technology includes methods of manufacturing the sensor. For instance, a method of manufacturing the sensor may include providing a substrate. Next, the method may include disposing the substrate in a seed solution. It is also contemplated for the seed solution to be disposed onto the substrate. The seed solution may include zinc. The seed solution may be configured to produce a zinc oxide seed layer on the substrate. Then, the substrate with the zinc oxide seed layer may be disposed into a growth solution. The growth solution may be configured to form a semiconducting nanostructured zinc oxide catalyst on the substrate. Afterwards, a semiconducting nanostructured zinc oxide catalyst may be formed on the substrate.

Further areas of applicability will become apparent from the description provided herein. It should be understood that the description and specific examples are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a box diagram of a sensor including a substrate and a piezoelectric semiconductor, according to one embodiment of the present disclosure;

FIG. 2 is a top view from a scanning electron microscope image of a zinc oxide nanorod, according to one embodiment of the present disclosure;

FIG. 3 is a top view from a scanning electron microscope image of a zinc oxide nanosheet, according to one embodiment of the present disclosure;

FIG. 4 is another top view from a scanning electron microscope image of a zinc oxide nanorod, according to one embodiment of the present disclosure;

FIG. 5 is a top view of an energy dispersive spectroscopy mapping image, further depicting a distribution of zinc within the zinc oxide nanorods, according to one embodiment of the present disclosure;

FIG. 6 is a top view of an energy dispersive spectroscopy mapping image, further depicting a distribution of oxide within the zinc oxide nanorods, according to one embodiment of the present disclosure;

FIG. 7 is another top view from a scanning electron microscope image of a zinc oxide nanosheet, according to one embodiment of the present disclosure;

FIG. 8 is a top view of an energy dispersive spectroscopy mapping image, further depicting a distribution of zinc within the zinc oxide nanosheets, according to one embodiment of the present disclosure;

FIG. 9 is a top view an energy dispersive spectroscopy mapping image, further depicting a distribution of oxide within the zinc oxide nanosheets, according to one embodiment of the present disclosure;

FIG. 10 is a line graph illustrating an x-ray diffraction pattern of zinc oxide nanorods (ZnO NRs) and zinc oxide nanosheets (ZnO NSs), according to one embodiment of the present disclosure;

FIG. 11 is a line graph illustrating nitrogen adsorption and desorption isotherms of ZnO NRs and NSs, according to one embodiment of the present disclosure;

FIG. 12 is a top perspective view of a finite element method simulation image illustrating a piezoelectric potential distribution of a bottom-fixed and electrically-grounded ZnO NR under an axial force, according to one embodiment of the present disclosure;

FIG. 13 is a top perspective view of a finite element method simulation image illustrating a piezoelectric potential distribution of a bottom-fixed and electrically-grounded ZnO NR under a lateral force, according to one embodiment of the present disclosure;

FIG. 14 is a front elevational view of a finite element method simulation image illustrating a piezoelectric potential distribution of a bottom-fixed and electrically-grounded ZnO NR under an axial force, according to one embodiment of the present disclosure;

FIG. 15 is a top perspective view of a finite element method simulation image illustrating a piezoelectric potential distribution of a ZnO NS under an axial force, according to one embodiment of the present disclosure;

FIG. 16 is a top perspective view of a finite element method simulation image illustrating a piezoelectric potential distribution of a ZnO NS under a lateral force, according to one embodiment of the present disclosure;

FIG. 17 is a front elevational view of a finite element method simulation image illustrating a piezoelectric potential distribution of a ZnO NS under a lateral force, according to one embodiment of the present disclosure;

FIG. 18 is a schematic diagram of a ZnO catalyst without polarization, according to one embodiment of the present disclosure;

FIG. 19 is a schematic diagram of a ZnO catalyst with electrocatalysis enhanced by a piezoelectric effect, further depicting a surface reaction under band tilting and strain, according to one embodiment of the present disclosure;

FIG. 20 is a front elevational view of a photograph illustrating an experimental setup of a piezoelectric ZnO/ITO-PET enhanced electrocatalysis of L-ascorbic acid (AA), according to one embodiment of the present disclosure;

FIG. 21 is a line graph illustrating the electrocatalysis of AA under 0.4% tensile strain in the presence of ZnO NRs and a control circumstance; according to one embodiment of the present disclosure;

FIG. 22 is a line graph illustrating the electrocatalysis of AA under 0.4% tensile strain in the presence of ZnO NSs and a control circumstance; according to one embodiment of the present disclosure;

FIG. 23 is a line graph illustrating the electrochemical impedance spectroscopy response of ZnO NRs and NSs, according to one embodiment of the present disclosure;

FIG. 24 is a bar graph illustrating the change rate of the reaction kinetics of AA catalysis before and after deformation of ZnO NRs and NSs, according to one embodiment of the present disclosure;

FIG. 25 is plot diagram illustrating a detection range width (mM) versus sensitivity (μA mM⁻¹ cm⁻²) of the piezoelectric ZnO NRs and NSs (under 0.4% strain) compared with various known catalysts;

FIG. 26 is a line graph illustrating the electrocatalysis of AA of ZnO NRs under different strain (0.2%, 0.4% and 0.6%), according to one embodiment of the present disclosure;

FIG. 27 is a line graph illustrating the electrocatalysis of AA of ZnO NSs under different strain (0.2%, 0.4% and 0.6%), according to one embodiment of the present disclosure;

FIG. 28 is a bar graph illustrating the reaction kinetics (ΔI=kC) of ZnO NRs and ZnO NSs under different strain (0.2%, 0.4% and 0.6%), according to one embodiment of the present disclosure;

FIG. 29 is a line graph illustrating the electrocatalysis of AA by 0.4% deformed ZnO NRs, according to one embodiment of the present disclosure;

FIG. 30 is a line graph illustrating the electrocatalysis of AA by 0.4% deformed NSs in the presence of different radical's scavengers, according to one embodiment of the present disclosure;

FIG. 31 is a bar graph illustrating the reaction kinetics (ΔI=kC) of 0.4% deformed ZnO NRs and NSs before and after adding different radical's scavengers, according to one embodiment of the present disclosure;

FIG. 32 is a front elevational, cross-sectional view of a scanning electron microscope image illustrating ZnO NRs, according to one embodiment of the present disclosure;

FIG. 33 is a top-plan view of an atomic-force microscopy image illustrating ZnO NSs, according to one embodiment of the present disclosure;

FIG. 34 is a line scan illustrating width and height parameters of the ZnO NSs along the line shown in FIG. 33, according to one embodiment of the present disclosure;

FIG. 35 is a line graph illustrating a room-temperature Raman spectra of ZnO NRs (blue) and ZnO NSs, according to one embodiment of the present disclosure;

FIG. 36 is a line graph illustrating a chronoamperograms of a ZnO NRs/ITO-PET electrode under 0.4% strain in 0.01 M NaOH solution at the potential of 0.4 V vs. Ag/AgCl, according to one embodiment of the present disclosure;

FIG. 37 is a line graph illustrating an amperometric response of the ZnO NRs/ITO-PET electrode under 0.4% strain at 0.4 V with additions of ascorbic acid and 2.0 mM different interfering species in 0.01 M NaOH solution, according to one embodiment of the present disclosure;

FIG. 38 is a table illustrating a performance comparison between the present disclosure and known electrochemical techniques for the detection of ascorbic acid;

FIG. 39 is a table illustrating a comparison of the sensing performance between the present disclosure and of different detection methods for the determination of ascorbic acid; and

FIG. 40 is a flow chart illustrating a method of manufacturing the sensor, according to one embodiment of the present disclosure.

### DETAILED DESCRIPTION

The following description of technology is merely exemplary in nature of the subject matter, manufacture, and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature unless otherwise disclosed, and thus, the order of the steps can be different in various embodiments, including where certain steps can be simultaneously performed.

### I. Definitions

Unless otherwise defined, all technical terms used herein have the same meaning as commonly understood by one of ordinary skill in the art to which the present disclosure pertains.

As used herein, the terms “a” and “an” indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters. In the present disclosure the terms “about” and “around” may allow for a degree of variability in a value or range, for example, within 10%, within 5%, or within 1% of a stated value or of a stated limit of a range. Likewise, in the present disclosure the term “substantially” can allow for a degree of variability in a value or range, for example, within 90%, within 95%, or within 99% of a stated value or of a stated limit of a range.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping, or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below,” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

### II. Description

Piezocatalysis emerges as an effective mechanism to enhance the efficiency of catalytic processes with the strain-induced piezoelectric field. In this case, the chemical species such as pollutants, dyes, drug, and H₂O molecules can thermodynamically undergo reduction or oxidation reactions when being in contact with the piezoelectric materials. The efficiency of a piezocatalysis process can be modulated by engineering the type, size, and morphology of the piezoelectric materials and controlling the mechanical stimuli (e.g., strain, pressure, etc.). Compared to traditional piezoelectric materials that are brittle and insulating, low-dimensional piezoelectric semiconductors such as zinc oxide nanowires and 2D transition-metal-dichalcogenides offer unexplored possibilities for leveraging the coupling of piezoelectricity to various catalytic processes. The superior mechanical properties of these nanostructured piezoelectrics also allow mechanical tunability inaccessible to bulk or thin-film materials, facilitating the efficient piezocatalysis driven by a low mechanical budget. Rationally designed, catalytically active, nanostructured piezoelectric semiconductors hold promise to address the challenges facing the current catalysts for the enhanced sensing of AA through cost-effective electrocatalytic pathways, e.g., with mechanical stimuli.

Advantageously, the electrochemical sensor system may provide a cost-efficient, high-performance piezo-electrocatalytic sensor for detecting AA, with the electrocatalytic efficacy significantly boosted by the piezoelectric polarization charges induced in the nanostructured semiconducting zinc oxide (ZnO) catalyst. Zinc oxide nanorods (ZnO NRs) 104 and nanosheets (NSs 106) 106 were prepared to characterize and compare their efficacy for the piezo-electrocatalysis of AA. The distribution of piezoelectric potential in the nanostructured ZnO catalysts 104, 106 were simulated using the finite element method (FEM). The relationship between the piezoelectric potential and piezocatalytic efficiency was established by elucidating the charge transfer between the strained ZnO nanostructures and AA. In a specific, non-limiting example, the deformed ZnO NRs 104 and NSs 106 possess boosted catalytic efficiency for AA, which increases around 4.72 times and around 0.5 times compared with that of the undeformed ZnO NRs 104 and NSs 106, respectively. With continued reference to the non-limiting example, the fabricated AA sensors exhibited wide actual detection ranges (10 μM-2.9 mM for deformed ZnO NRs 104 and 10 μM-3.4 mM for deformed ZnO NSs 106) and low detection limits (0.48 μM for deformed ZnO NRs 104 and 0.72 μM for deformed ZnO NSs 106, S/N=3), superior to the state-of-the-art AA sensors. It is contemplated the piezo-electrocatalytic process of the present disclosure could utilize the otherwise wasted environmental mechanical energy (e.g., wind energy, wave energy, tidal energy, biomechanical energy, etc.) to boost the electrocatalytic efficiency. The concept of piezo-electrocatalysis can be extended to numerous other catalytic processes of biomedical, pharmaceutical, and agricultural interest. Desirably, the hydrothermal synthesis of ZnO nanostructures also allows the low-cost, scalable production and integration of piezoelectrically-enhanced AA sensors into deformable form factors for wearable sensors capable of real-time and non-invasive monitoring of uric acid, lactate, ascorbic acid, glucose, and caffeine in sweat, where the sensor performance could be boosted by the human-generated mechanical signals.

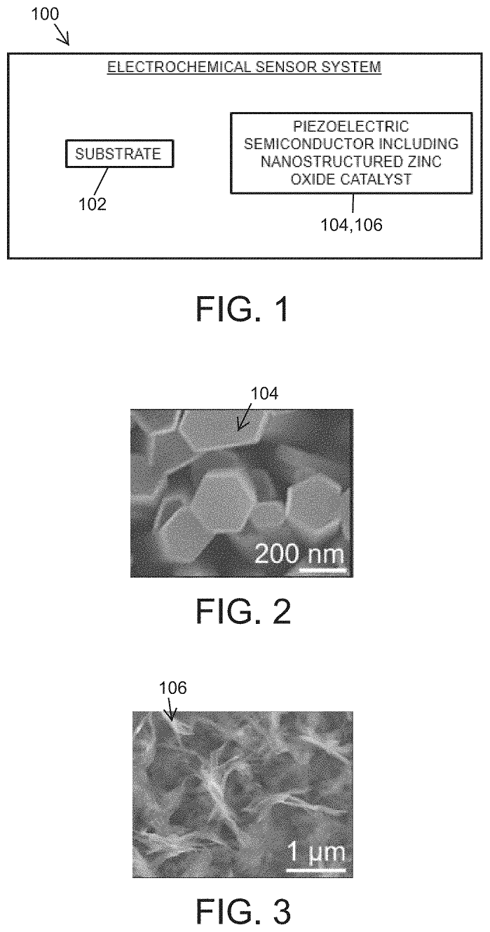

The sensor 100 is configured to detect ascorbic acid using piezo-electrocatalysis. As shown in FIG. 1, the sensor 100 includes a substrate 102 and a piezoelectric semiconductor 104, 106. The piezoelectric semiconductor 104, 106 may be coupled to the substrate 102. In a specific example, the piezoelectric semiconductor 104, 106 may be hydrothermally synthesized to the substrate 102. In a specific example, the piezoelectric semiconductor 104, 106 may include a nanostructured semiconducting ZnO catalyst. In certain circumstances, the nanostructured semiconducting ZnO catalyst 104, 106 may have a noncentrosymmetric wurtzite configuration.

The nanostructured semiconducting ZnO catalyst 104, 106 may be provided in many ways. For instance, the nanostructured semiconducting ZnO catalyst 104, 106 may have a noncentrosymmetric wurtzite configuration. The noncentrosymmetric wurtzite configuration may include a crystal formation with hexagonal symmetry. In a specific example, the nanostructured semiconducting ZnO catalyst 104, 106 may be provided as a ZnO nanorod 104. The ZnO nanorod 104 may have a terminal end 108 connected to the substrate 102. The ZnO nanorod 104 may have a substantially hexagonal cross-section. In another specific, non-limiting example, the nanostructured semiconducting ZnO catalyst 104, 106 may be provided as a ZnO nanosheet 106. A skilled artisan may select other suitable shapes and formations of the ZnO catalyst 104, 106, within the scope of the present disclosure.

In certain circumstances, the substrate 102 may be provided in various ways. For instance, the substrate 102 may be constructed from a conductive material, such as a metal material and/or graphite. In a more specific example, the substrate 102 may include an indium tin oxide substrate. In an even more specific example, the substrate 102 may include an indium tin oxide coated polyethylene terephthalate film. One skilled in the art may select other suitable materials to form the substrate 102, within the scope of the present disclosure.

In certain circumstances, the sensor 100 may be configured with various capabilities and applications. For instance, the sensor 100 may have a limit of detection less than three micromolars. Limit of Detection may be understood as the lowest quantity or concentration of a component that can be reliably detected with a given analytical method. Advantageously, the present disclosure is capable of lower limits of detection compared to known sensors, thereby enhancing the accuracy and fields of application of the sensor 100. As a non-limiting example, the sensor 100 may be provided in a wearable electrocatalytic device. In a specific example, the sensor 100 may also detect uric acid, lactate, glucose, and/or caffeine. In as further non-limiting examples, the sensor 100 may be configured to be utilized in many applications such as biomedical devices, pharmaceutical devices, and agricultural devices.

In certain circumstances, the nanostructured semiconducting ZnO catalyst 104, 106 may be capable of inducing piezoelectric polarization charges while under mechanical deformations. As a non-limiting example, the mechanical deformation may include an applied force that pushes the ZnO catalyst 104, 106 causing a bending curvature between around 0.2% to around 0.6%. In a specific example, the piezoelectric potential in a nanorod 104 may continuously distribute along a polar axis where an axial compression is applied to the ZnO nanorod 104 with its terminal end 108 mechanically fixed to the substrate 102 and electrically grounded. As shown in FIG. 12, the piezoelectric potential in the nanorod 104 continuously distributes along the polar axis.

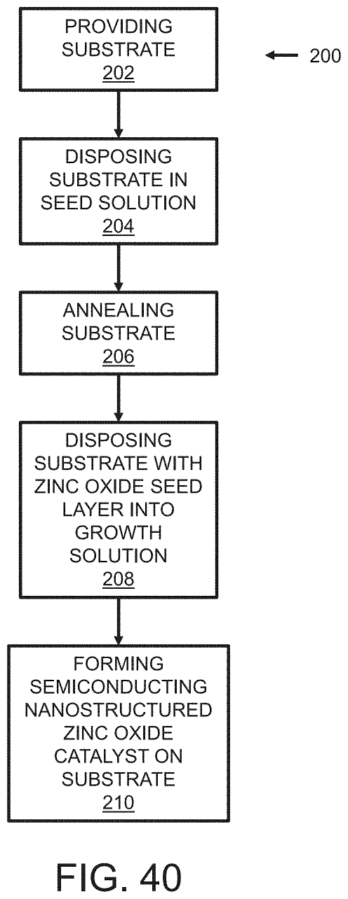

In another embodiment, the present technology includes methods of manufacturing the sensor 100. For instance, as shown in FIG. 40, a method 200 of manufacturing the sensor 100 may include providing a substrate 102. The substrate 102 may be constructed from a conductive material, such as a metal material and/or graphite. In a more specific example, the substrate 102 may include an indium tin oxide substrate. In an even more specific example, the substrate 102 may include an indium tin oxide coated polyethylene terephthalate film. Next, the method 200 may include disposing the substrate 102 in a seed solution. It is also contemplated for the seed solution to be disposed onto the substrate 102. The seed solution may include a zinc salt such as zinc acetate, zinc nitrate, and/or zinc chloride. In a more specific example, the seed solution may include zinc acetate dihydrate. The seed solution may be configured to produce a ZnO seed layer on the substrate 102. In some circumstances, the method 200 may include a step 206 of annealing the substrate 102. In a specific example, the substrate 102 may be annealed at around eight-five degree Celsius. In a more specific example, the substrate 102 may be disposed in the seed solution and annealed multiple times. In an even more specific example, the substrate 102 may undergo a final annealing cycle at around two-hundred fifty degrees Celsius for around twenty minutes. After the substrate 102 is annealed, a ZnO seed layer may be coupled to the substrate 102. Then, the substrate 102 with the ZnO seed layer may be disposed into a growth solution. The growth solution may be configured to form a semiconducting nanostructured ZnO catalyst 104, 106 on the substrate 102. In a specific example, the growth solution may include zinc nitrate and/or hexamethylenetetramine to form ZnO NRs 104. In a separate, specific example, the growth solution may include zinc chloride and potassium chloride to form ZnO NSs 106. Afterwards, a semiconducting nanostructured ZnO catalyst 104, 106 may be formed on the substrate 102, thereby manufacturing the sensor 100.

### III. Example

Provided as a specific, non-limiting example, one embodiment of the ZnO NRs 104 and NSs 106 were synthesized via a hydrothermal method. The hydrothermal method may be understood as the process of crystallizing substances from high-temperature aqueous solutions at high vapor pressures. The morphology control was achieved through the surface selective electrostatic interaction. The growth solutions of the two morphologies consist of different zinc-precursors, namely zinc nitrate and zinc chloride. The introduction of Cl⁻ benefits the growth of nanosheets 106. Without being bound to any particular theory, it is believed the selective adsorption of the highly electronegative Cl⁻ ions on a polar ±(0001) plane may hinder the growth of the ZnO nanostructure along a polar axis [0001] direction.

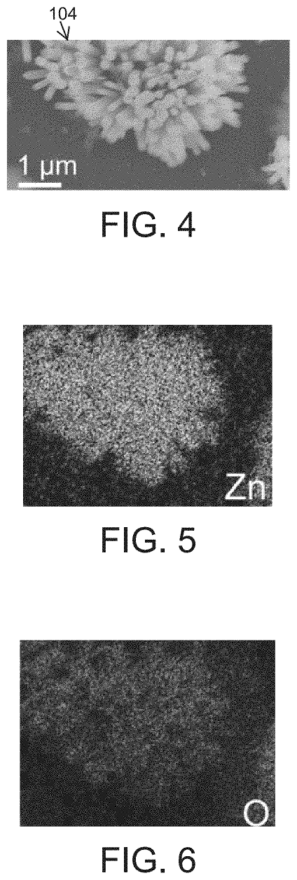

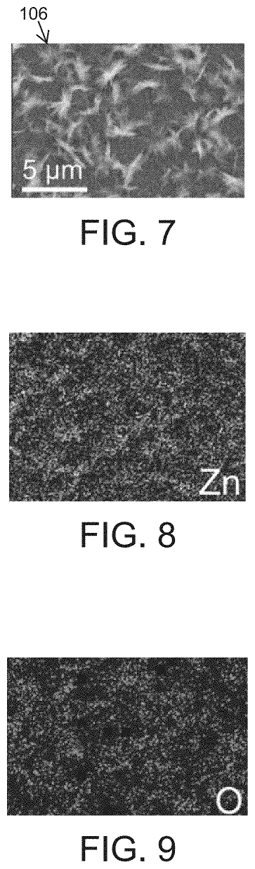

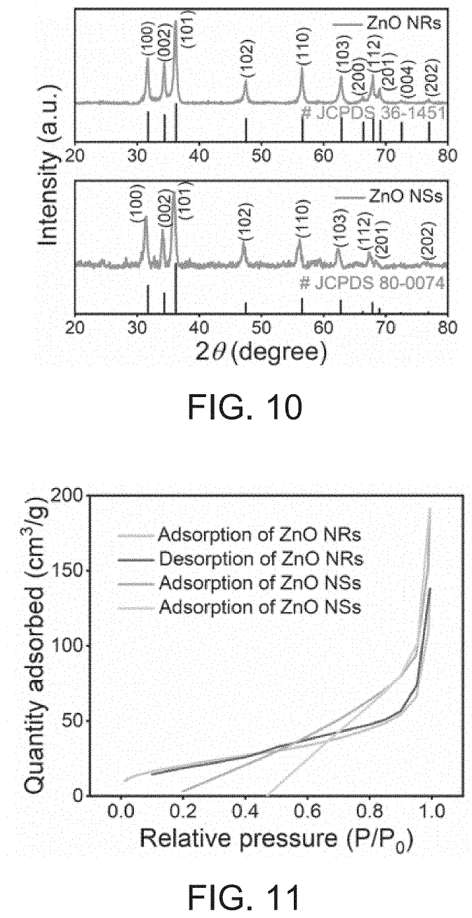

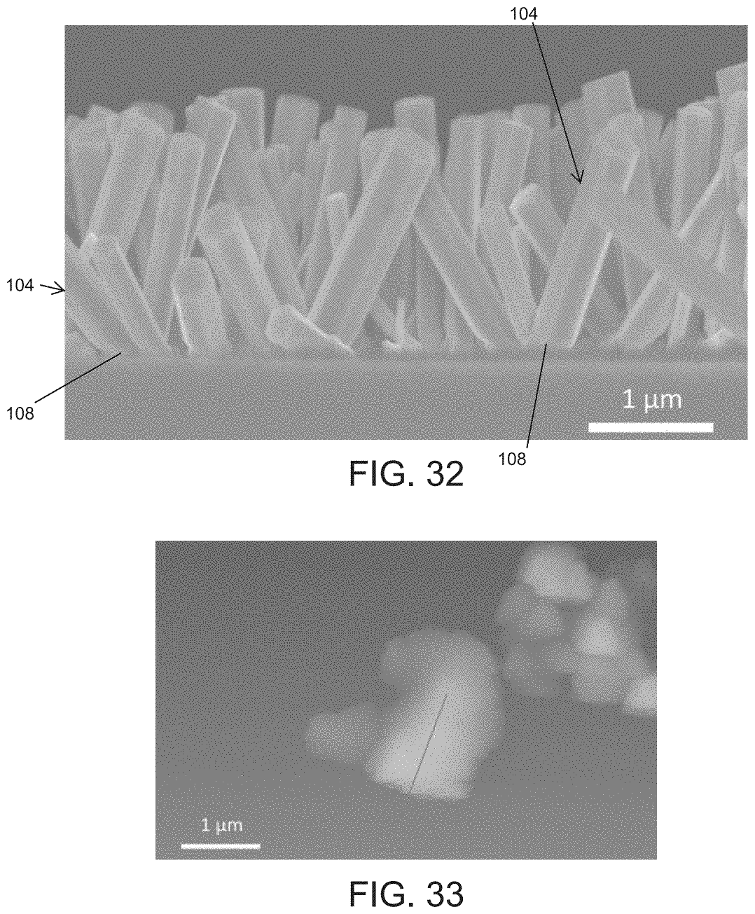

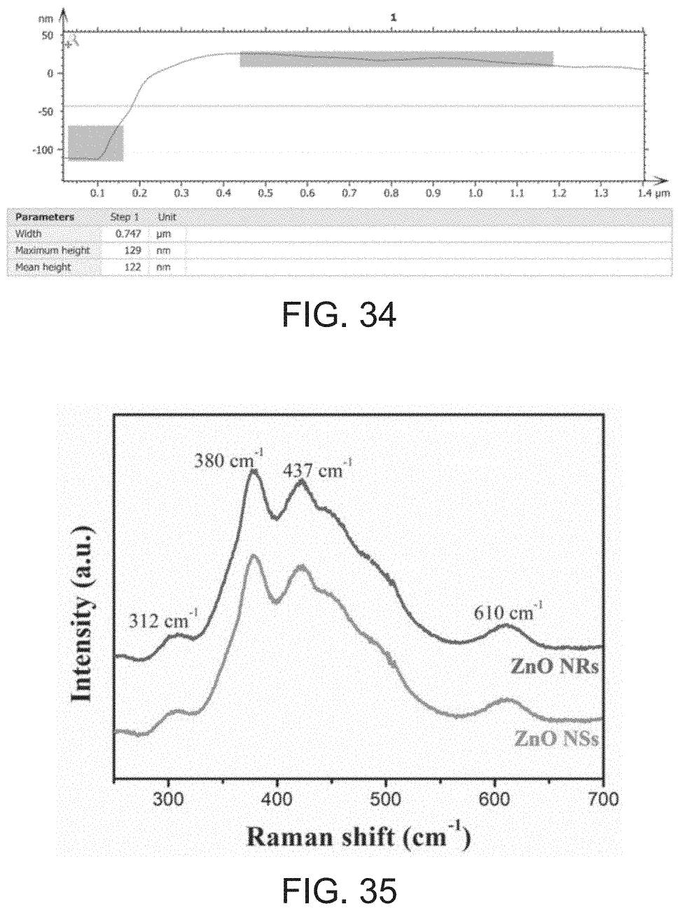

The morphologies of the as-synthesized ZnO NRs 104 and NSs 106 on indium tin oxide (ITO) substrates 102 were examined using scanning electron microscopy (SEM), as shown in FIGS. 2-9 and 32. The obtained ZnO NRs 104 exhibit a homogenous rod-like morphology (with a uniform diameter of ˜200 nm and a length of ˜2 μm) and a hexagonal cross-section typical to the one-dimensional wurtzite ZnO structures. The SEM and atomic force microscope (AFM) characterizations of ZnO NSs 106, as shown in FIGS. 3, 7-9, and 33-34 revealed their 2D morphology, with an average thickness of ˜122 nm and an edge length ˜1-2 μm. The energy-dispersive X-ray spectroscopy (EDS) mapping, as shown in FIGS. 4-9, confirmed that the obtained samples consist of zinc and oxygen elements. The crystal structure and phase purity of the ZnO NRs 104 and NSs 106 were examined with X-ray diffraction (XRD) analysis, as shown in FIG. 10. As a non-limiting reference, the as-synthesized ZnO NRs 104 were identified with the ZnO hexagonal wurtzite structure (JCPDS Card No. 36-1451), and the ZnO NSs 106 were identified with the wurtzite ZnO crystalline phase (JCPDS Card No. 80-0074). The sharp diffraction peaks also indicate good crystallinity of the obtained samples. Raman spectra were also measured to study the lattice vibration of ZnO NRs 104 and NSs 106, as shown in FIG. 35. The peak at 437 cm⁻¹ corresponds to the non-optical phonon E_{2H} mode and is associated with the oxygen sublattice, confirming the wurtzite structure of both the ZnO NRs 104 and NSs 106. No other impurity peaks appeared in the XRD and Raman spectra, indicating the formation of pure phase materials.

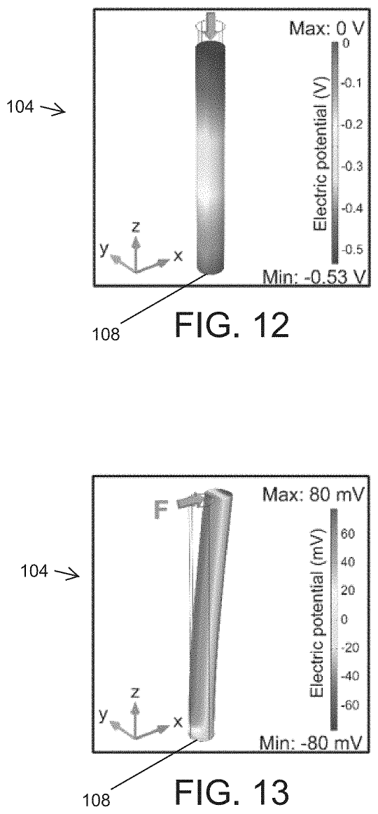

The specific surface area of the obtained ZnO nanomaterials were characterized by N₂ adsorption-desorption analysis, as shown in FIG. 11. According to the Brunauer-Emmett-Teller (BET) model, the specific surface area of ZnO NRs 104 can be determined as 75.8 m² g⁻¹, which is approximately 4.6 times higher than that of ZnO NSs 106 (13.6 m² g⁻¹). Such a difference in the specific surface area can be understood because NRs 104 extend more in the z-axis and are densely arranged, resulting in more exposed surfaces. The nanorods 104 with a high surface area provide a larger amount of accessible reactive sites for the piezocatalysis of AA, facilitating the adsorption and desorption of AA and facilitate the efficient electric charge transfer between the catalyst and reactant.

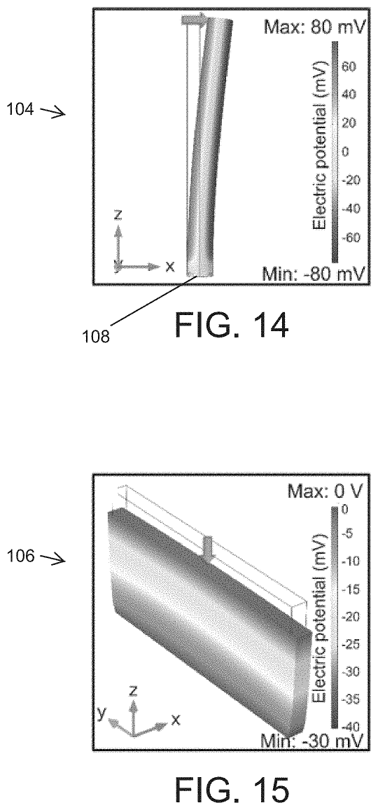

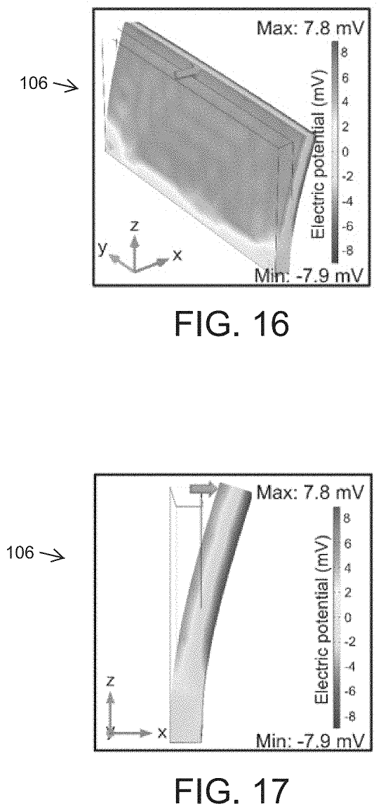

ZnO with a noncentrosymmetric wurtzite structure can induce piezoelectric polarization charges on the surface under mechanical deformations. A finite element calculation was performed to simulate the distribution of piezoelectric potential produced in the ZnO NRs 104 and NSs 106, as shown in FIGS. 12-17. In all simulations, the polar axis (i.e., [0001]) of the ZnO NRs 104 were oriented along the z-axis. As shown in FIG. 12, the FEM simulation results for the case when an axial compression was applied to a ZnO nanorod 104 with the terminal end 108 mechanically fixed to the substrate 102 and electrically grounded. The simulation indicates that the piezoelectric potential in the nanorod 104 continuously distributes along the polar axis. As shown in FIGS. 13-14 and 16-17, both the ZnO NRs 104 and NSs 106 can be facilely deformed when a lateral force was applied along the upper surface of the material. In particular, a positive potential is generated on the stretched side of the nanorod 104, and a negative potential is generated on the compressed side of the nanorod 104. The dimension values measured from the material characterization in the FEM simulation were inputted. For ZnO NRs 104 with a diameter (D)=200 nm, length (L)=2 μm, and force (F)=80 nN, FEM simulation shows that the maximum piezoelectric potential generated is 160 mV, as shown in FIGS. 13-14. For ZnO NSs 106 with L=2 μm, width=122 nm, height=1 μm, and F=80 nN, the induced piezoelectric potential is 15.7 mV. The difference between the induced piezoelectric potential in ZnO NRs 104 and NSs 106 may be understood by the different aspect ratios for these two morphologies, which leads to significantly different deformation behaviors. Because a single nanorod 104 has a larger aspect ratio (length/diameter, ca. 10:1) and a smaller ground contact area than nanosheet 106, it can be easily deformed. Without being bound to any particular theory, it is believed that a higher piezoelectric potential will result in a more efficient catalytic process.

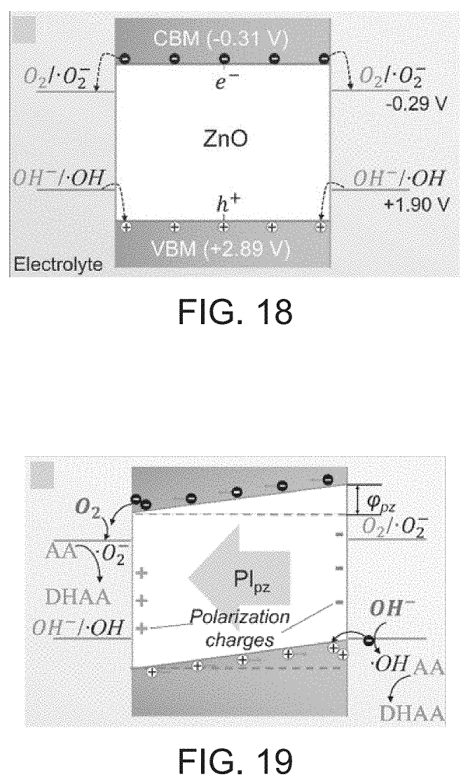

The working mechanism of the piezo-electrocatalysis was further explored by examining the related band diagram, as shown in FIGS. 18-19. The standard redox potential of O₂/·O₂⁻ (−0.29 V) is more positive than the conduction band minimum (CBM) (−0.31 V) of ZnO, as shown in FIG. 18, thus allowing the reduction of dissolved O₂ by the conduction band (CB) electrons from ZnO to produce ·O₂⁻ radicals. Meanwhile, the standard redox potential of OH⁻/·OH (+1.90 V) is more negative than the valence band maximum (VBM) of ZnO (+2.89 V), resulting in the transport of holes to the electrolyte and, therefore, the oxidation of OH⁻ to ·OH radicals. When an external force is applied, the induced piezoelectric polarization charges on the surfaces of ZnO NRs 104 and NSs 106 couple with the electrical excitation in impacting the redox processes that occur on the ZnO surfaces. As illustrated in FIG. 19, the band tilting in ZnO with the piezoelectric potential further promotes the above two redox processes for O₂ and OH⁻ at the respective surfaces. Such an enhanced redox process occurs in ZnO NRs 104 and NSs 106 independent of the direction of the applied forces. The relatively larger piezoelectric potential in ZnO NRs 104 may lead to a more efficient generation of the ·O₂⁻ and ·OH radicals, exhibiting improved catalytic performance. Electrons in the conduction band can react with oxygen to create superoxide radicals (·O₂⁻), meanwhile, the holes generated at the top of the valance band are energetically favorable for the production of oxidative hydroxyl radicals (·OH). The redox process of AA on the ZnO electrode surface is shown below.

$$ZnO = \begin{matrix}Electrical - excitation \\ ⟶ \\ Piezoelectric effect\end{matrix} ZnO ( {h}^{+} + {e}^{-} ) \qquad ( 1 )$$

$${OH}^{-} + {h}^{+} ⟶ \cdot OH \qquad ( 2 )$$

$${O}_{2} + {e}^{-} ⟶ \cdot {O}_{2}^{-} \qquad ( 3 )$$

$$\cdot OH ( \cdot {O}_{2}^{-} ) + AA ( {C}_{6} {H}_{8} {O}_{6} ) ⟶ DHAA ( {C}_{6} {H}_{6} {O}_{6} ) \qquad ( 4 )$$

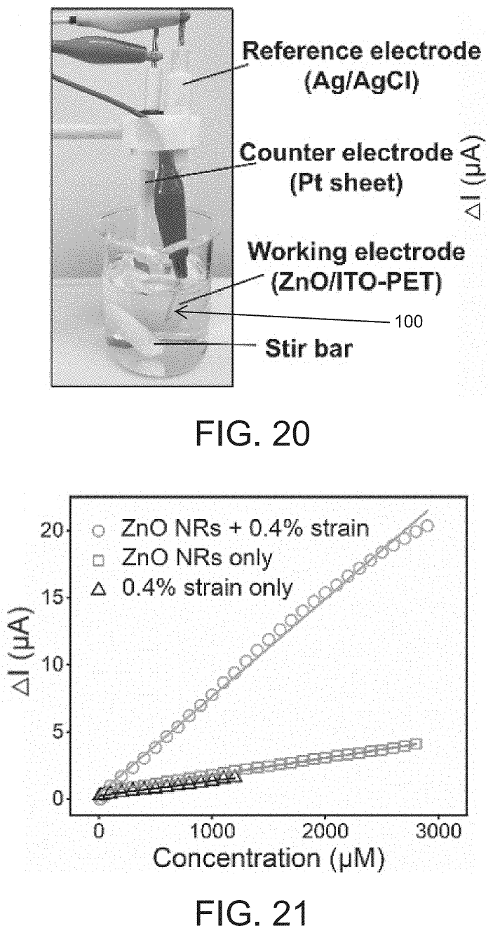

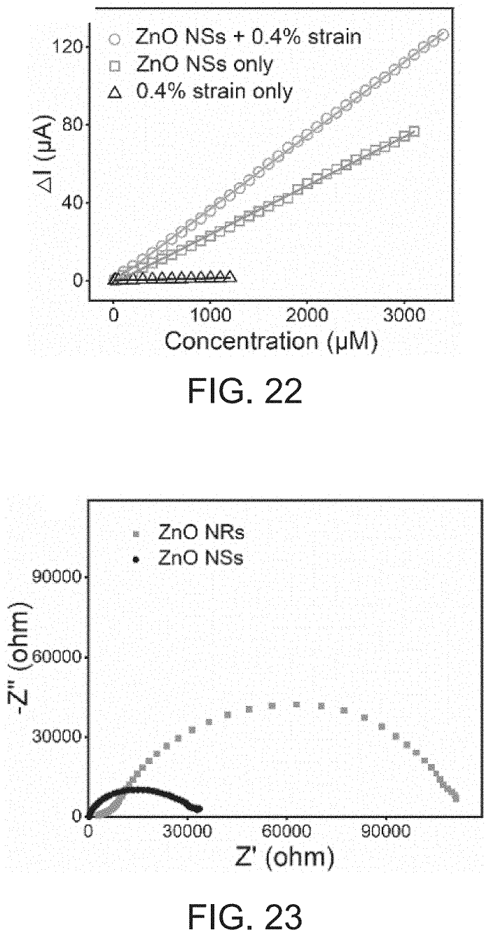

FIG. 20 illustrates the experimental setup for characterizing the piezo-electrocatalysis of AA. ZnO nanomaterials were hydrothermally synthesized on the ITO-PET substrate 102. The applied strain was controlled by modulating the bending curvature of the substrate 102. The catalytic efficiency of different electrodes was characterized by recording the catalytic currents for different concentrations of AA. The impact of the piezoelectric effect on the electrocatalytic sensing of AA was investigated under three different experimental conditions: undeformed ZnO nanostructures, deformed bare ITO-PET substrates 102, and deformed ZnO nanostructures on ITO-PET substrates 102, as shown in FIGS. 21-22. The experimental results show that under different experimental conditions, the current change (ΔI) increases with the increase of the AA concentration, indicating that these conditions can cause AA to be catalyzed. As shown in FIG. 21, the catalytical performance was enhanced only when both the substrate deformation and piezoelectric material exist simultaneously, indicated by open circles in FIG. 21. In contrast, the electrocatalysis of AA in the absence of either ZnO NRs 104 or deformation was almost negligible. The catalysis capability of undeformed ZnO NRs 104, indicated by open squares in FIG. 21, is attributed to the electrocatalytic property of semiconducting ZnO. The catalytic performance of deformed ZnO with 0.4% tensile strain is due to the combined electrocatalysis and piezocatalysis.

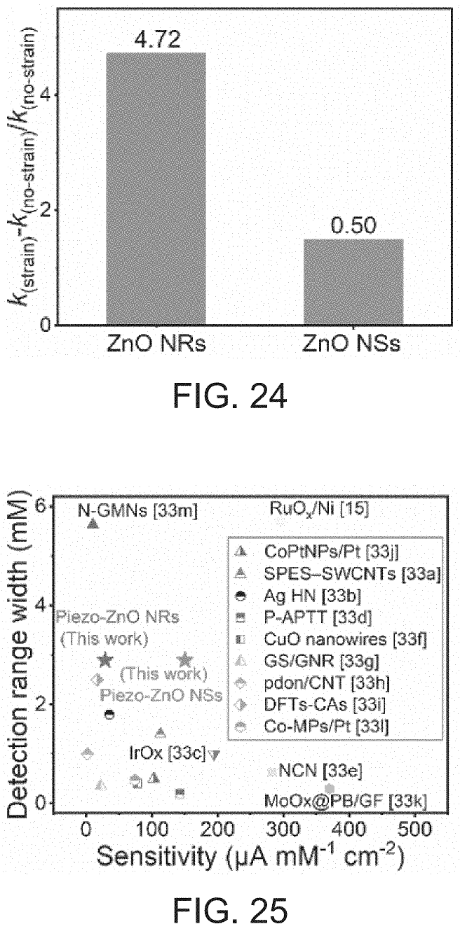

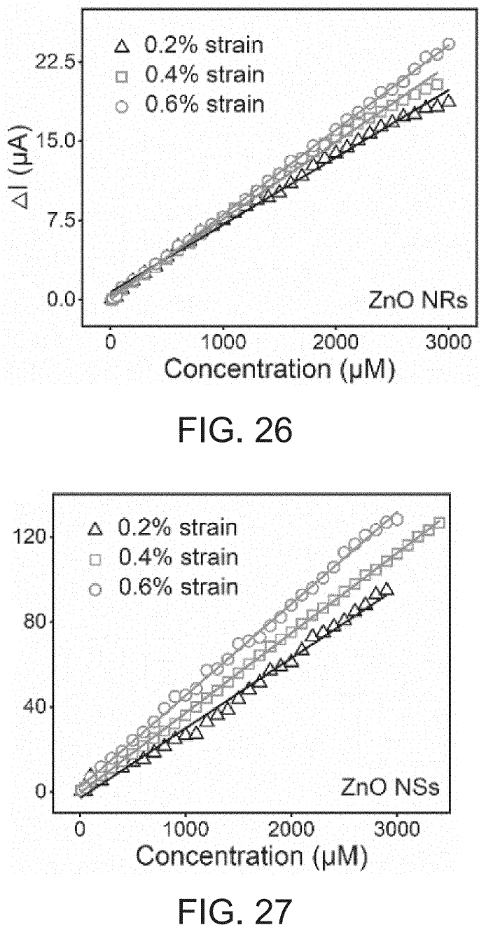

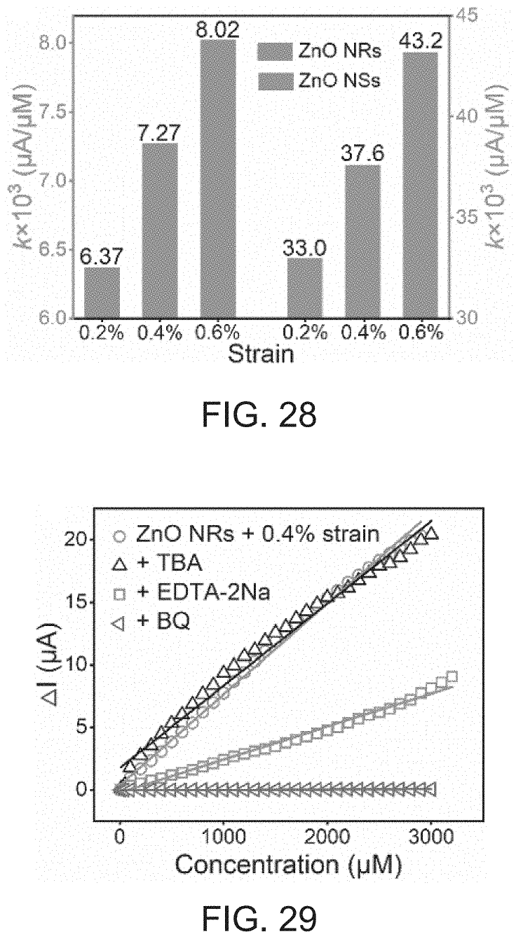

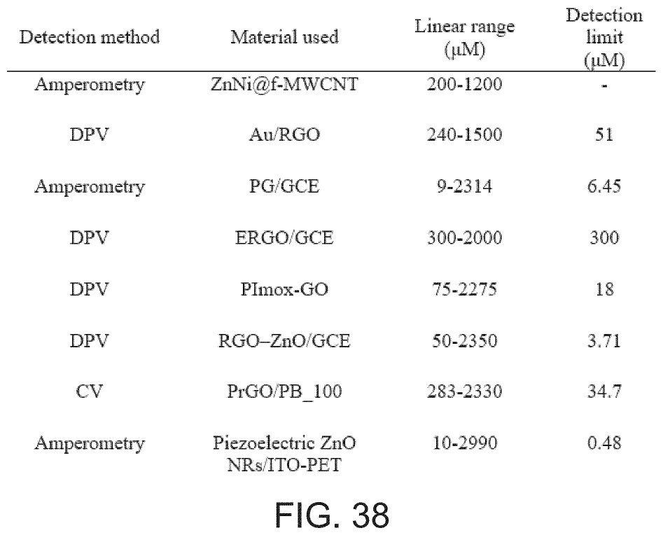

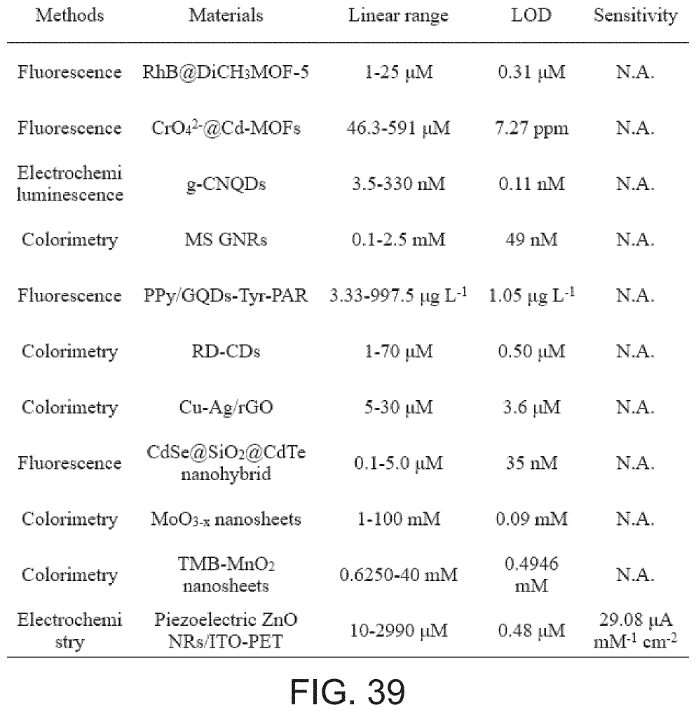

With continued reference to FIGS. 21-22, the reaction kinetics for the piezo-electrocatalysis of AA can be expressed as a linear correlation between ΔI and the AA concentration (C). The corresponding regression, detection range, and limit of detection (LOD) for different AA catalysts are compared in Table 1 on the following page. The LOD of deformed ZnO NRs 104 is calculated as 0.48 μM (S/N=3) and is lower than many known electrocatalysis sensors for AA, as shown in FIG. 38. At the same time, compared with other reported detection methods, piezoelectric ZnO nanomaterials also show comparable detection performance, as shown in FIG. 39. The kinetic rate constant k for the process in 0.4% deformed ZnO NRs 104 increases by 4.72 times compared to that for the undeformed ZnO NRs 104 (from 0.00127 to 0.00727 μA μM⁻¹), as shown in FIG. 21. Meanwhile, the undeformed ZnO NSs 106 have a relatively high background current due to the much lower charge transfer resistance (R_{ct}), as shown in FIG. 23. The kinetic rate constant k for the process in the deformed ZnO NSs 106 increases by 0.5 times compared to that for the undeformed ZnO NSs 106 (from 0.0251 to 0.0376 μA M⁻¹), as shown in FIG. 22. The strain-induced enhancement in the electrocatalytic sensing of AA can be determined by (k₍ₛₜᵣₐᵢₙ₎-k_{(strain-free)})/k_{(strain-free)}, where the kinetic rate constant k (μA M⁻¹) is the slope of the current change-concentration (ΔI-C) plot. The strain-induced enhancement factors for both ZnO NRs 104 and NSs 106 are shown in FIG. 24. The detection range and sensitivity (μA mM⁻¹ cm⁻²) of the ZnO piezo-electrocatalysts were compared to known AA catalysts, as shown in FIG. 25. The piezo-electrocatalytic sensing of AA exhibits good overall performance compared to the state-of-the-art, as illustrated in FIG. 25. Known catalysts that have competitive performance metrics (e.g., RuOₓ/Ni) are also known to face challenges such as expensive raw materials and complex preparation processes. Compared to ZnO NSs 106, ZnO NRs 104 present more significant piezoelectric enhancement in the electrocatalytic sensing of AA, consistent with our simulation results, as shown in FIGS. 12-17. The piezo-electrocatalytic sensing of AA with ZnO NRs 104 also show more appealing figures of merit, e.g., lower LOD, and comparable sensitivity and detection range, as shown in FIG. 25 and Table 1 below, compared to ZnO NSs 106. It should be appreciated, that the sensing processes (with and without strains) with ZnO NSs 106 show higher ΔI values than ZnO NRs 104, as shown in FIGS. 21-22, which is because the higher conductivity of ZnO NSs 106, as shown in FIG. 23, can accelerate the oxidation reaction of AA on the surface of the material. We further characterized the piezo-electrocatalytic process in ZnO NRs 104 and NSs 106 based electrodes under different strains, as shown in FIGS. 26-27. The rate constant k increased as the strain increased, as shown in FIG. 28. The relationship between the rate constant and the strains indicates that a more significant piezoelectric effect induced by larger strains induced more significant catalytic enhancement.

TABLE 1
Regression, detection range, and limit of detection of different catalyst

| | | | LOD |
|---|---|---|---|
| Catalyst | Linear regression | Detection range | (S/N = 3) |
| ZnO NRs + strain | ΔI (μA) = 0.00727 C + 0.366 (R² = 0.9957) | 10 μM-2.9 mM | 0.48 μM |
| ZnO NRs only | ΔI (μA) = 0.00127 C + 0.525 (R² = 0.9988) | 10 μM-2.8 mM | 2.76 μM |
| ZnO NSs + strain | ΔI (μA) = 0.0376 C − 0.492 (R² = 0.9997) | 10 μM-3.4 mM | 0.72 μM |
| ZnO NSs only | ΔI (μA) = 0.0251 C − 1.11 (R² = 0.9989) | 10 μM-3.1 mM | 1.13 μM |
| Strain only | ΔI (μA) = 0.0011 C + 0.248 (R² = 0.9965) | — | — |

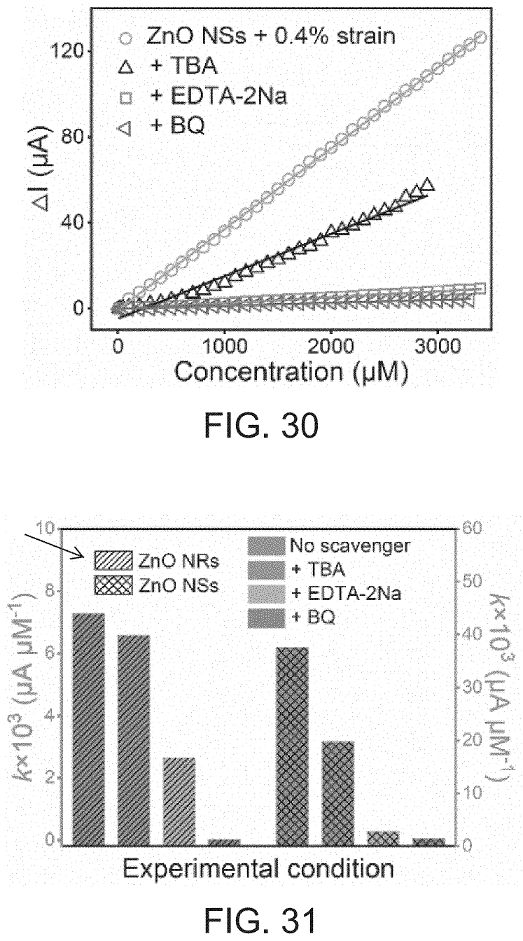

A series of radical trapping experiments were performed using various radical scavengers to identify the role of the radical species and elucidate the fundamental processes involved in the piezo-electrocatalytic sensing of AA. To this end, benzoquinone (BQ), tert-butyl alcohol (TBA), and disodium ethylene diamine tetraacetate dehydrates (EDTA-2Na) were used to scavenge superoxide radicals (·O₂⁻), hydroxyl (·OH), and holes (h⁺), respectively. It is believed that these radicals are induced by the piezoelectric effect. FIGS. 29-30 show the ΔI-C plots for 0.4% strained ZnO NRs 104 and NSs 106 with these radical scavengers. The rate constants for these processes are listed in FIG. 31. The catalytical efficiency of ZnO NRs 104 was remarkably suppressed by BQ (·O₂⁻ scavenger) and EDTA-2Na (h⁺ scavenger), which decreased by 99.73% and 63.69%, respectively. With the addition of ·OH radical scavenger of TBA (·OH scavenger), the catalytical ratio of AA was slightly reduced by 9.49%, illustrating that ·O₂⁻ radicals and holes are the main active species in the piezo-electrocatalytic process. These scavengers also show a similar inhibitory effect on the ZnO NSs 106-based piezo-electrocatalytic activity, as shown in FIGS. 30-31. After adding these inhibitors (TBA, EDTA-2Na, BQ), the catalytic efficiency for AA was reduced by 47.45%, 92.69% and 96.25%, respectively, for ZnO NSs 106. These results indicate that the piezoelectric effect leads to the generation of ·OH, ·O₂⁻ radicals and holes in situ on the surface of both ZnO NRs 104 and NSs 106, which will further react with AA molecules to accelerate the catalytical process (Equations 1-4).

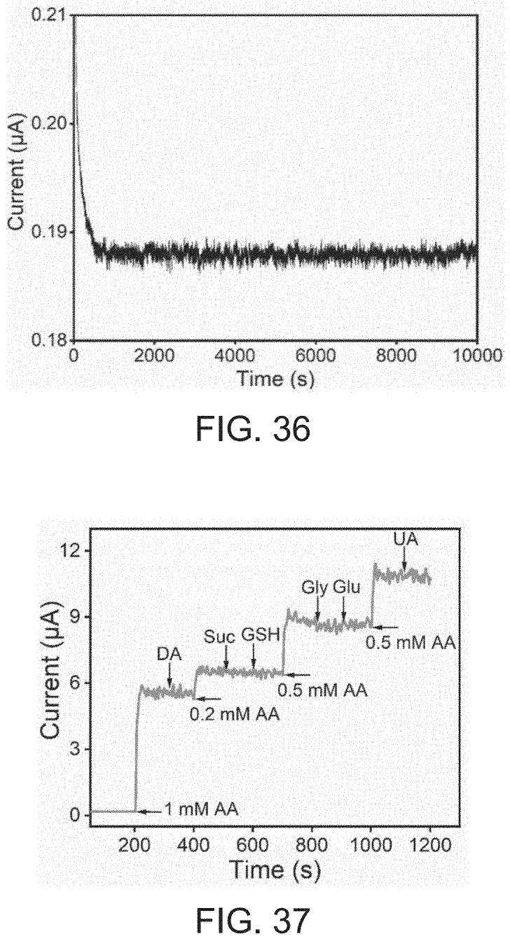

In addition to the catalytic activity, catalytic stability is another critical factor in evaluating reliable electrocatalysts for AA detection. In order to measure the material tolerance and long-term catalytical stability of the ZnO catalysts 104, 106 in the AA detection environment, chronoamperometric measurements were performed for deformed ZnO NRs 104 in 0.01 M NaOH solution at 0.4 V vs. Ag/AgCl for a duration of 10,000 s. The result shows no observable degradation in the piezo-electrocatalytic performance of ZnO NRs 104 after over one and half hours, as shown in FIG. 36. An anti-interference experiment was performed by adding different concentrations of AA and 2.0 mM interfering substances (dopamine (DA), Suc (sucrose), GSH (glutathione), Gly (glycine), Glu (glucose), UA (uric acid)) in a buffer solution sequentially. The result shows that the sensor 100 of present disclosure may only have a significant current response to AA, showing excellent anti-interference performance, as shown in FIG. 37.

Advantageously, the sensor 100 may provide a cost-efficient, high-performance piezo-electrocatalytic sensor for detecting AA, with the electrocatalytic efficacy significantly boosted by the piezoelectric polarization charges induced in the nanostructured semiconducting zinc oxide (ZnO) catalyst 104, 106.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions, and methods can be made within the scope of the present technology, with substantially similar results.

What is claimed is:

1. An electrochemical sensor configured to detect ascorbic acid using piezo-electrocatalysis, wherein the sensor comprises:
a substrate; and
a piezoelectric semiconductor coupled to the substrate, wherein the piezoelectric semiconductor includes a nanostructured semiconducting zinc oxide catalyst.

2. The electrochemical sensor of claim 1, wherein the nanostructured semiconducting zinc oxide catalyst has a noncentrosymmetric wurtzite configuration.

3. The electrochemical sensor of claim 2, wherein the nanostructured semiconducting zinc oxide catalyst is capable of inducing piezoelectric polarization charges while under mechanical deformations.

4. The electrochemical sensor of claim 1, wherein the nanostructured semiconducting zinc oxide catalyst is a zinc oxide nanorod.

5. The electrochemical sensor of claim 1, wherein the zinc oxide nanorod has a terminal end connected to the substrate.

6. The electrochemical sensor of claim 1, wherein the zinc oxide nanorod has a substantially hexagonal cross-section.

7. The electrochemical sensor of claim 1, wherein the nanostructured semiconducting zinc oxide catalyst is a zinc oxide nanosheet.

8. The electrochemical sensor of claim 1, wherein the substrate is constructed from a conducting material.

9. The electrochemical sensor of claim 1, wherein the substrate includes an indium tin oxide substrate.

10. The electrochemical sensor of claim 1, wherein the substrate includes an indium tin oxide coated polyethylene terephthalate film.

11. The electrochemical sensor of claim 1, wherein the piezoelectric semiconductor is hydrothermally synthesized to the substrate.

12. The electrochemical sensor of claim 1, wherein the sensor has a limit of detection less than three micromolars.

13. The electrochemical sensor of claim 1, wherein the sensor also detects at least one of uric acid, lactate, glucose, and caffeine.

14. A wearable electrocatalytic device comprising an electrochemical sensor according to claim 1.

15. A biomedical device comprising an electrochemical sensor according to claim 1.

16. A method of manufacturing an electrochemical sensor configured to detect ascorbic acid using piezo-electrocatalysis, the method comprising the steps of:
providing a substrate;
disposing the substrate in a seed solution including zinc, the seed solution configured to produce a zinc oxide seed layer on the substrate;
disposing the substrate with the zinc oxide seed layer into a growth solution, the growth solution configured to form a semiconducting nanostructured zinc oxide catalyst on the substrate; and
forming a semiconducting nanostructured zinc oxide catalyst on the substrate.

17. The method of claim 16, further comprising a step of annealing the substrate after the substrate was disposed in the seed solution but before the substrate is disposed into the growth solution.

18. The method of claim 16, wherein the seed solution includes a zinc salt.

19. The method of claim 16, wherein the growth solution includes at least one of zinc nitrate and hexamethylenetetramine.

20. The method of claim 16, wherein the growth solution includes at least one of zinc chloride and potassium chloride.
